SEQUENCE LISTING (54 sequences)

SEQ ID NO: 1 - protein, 13 aa, synthetic construct
CAETTLDFNKFYF

SEQ ID NO: 2 - protein, 15 aa, synthetic construct
CALSEETGANSKLTF

SEQ ID NO: 3 - protein, 13 aa, synthetic construct
CSEWWAAGNKLTF

SEQ ID NO: 4 - protein, 15 aa, synthetic construct
CALSDIGGATNKLIF

SEQ ID NO: 5 - protein, 15 aa, synthetic construct
CAVSDGGGSNYKLTF

SEQ ID NO: 6 - protein, 14 aa, synthetic construct
CATDASAAGNKLTF

SEQ ID NO: 7 - protein, 13 aa, synthetic construct
CAEIPINDYKLSF

SEQ ID NO: 8 - protein, 15 aa, synthetic construct
CGADGTSGSARQLTF

SEQ ID NO: 9 - protein, 15 aa, synthetic construct
CALSEAGGATNKLIF

SEQ ID NO: 10 - protein, 14 aa, synthetic construct
CATDATGGGNKLTF

SEQ ID NO: 11 - protein, 15 aa, synthetic construct
CAVSEGSGGYQKVTF

SEQ ID NO: 12 - protein, 15 aa, synthetic construct
CLVGESSSGTYKYIF

SEQ ID NO: 13 - protein, 13 aa, synthetic construct
CGEPLSGYNKLIF

SEQ ID NO: 14 - protein, 14 aa, synthetic construct
CGTEGITSGSRLTF

SEQ ID NO: 15 - protein, 13 aa, synthetic construct
CAEAGGGYQKVTF

SEQ ID NO: 16 - protein, 14 aa, synthetic construct
CAVEGTGGYQKVTF

SEQ ID NO: 17 - protein, 16 aa, synthetic construct
CAVSDHSSGSARQLTF

SEQ ID NO: 18 - protein, 13 aa, synthetic construct
CGEQGTGGFKTIF

SEQ ID NO: 19 - protein, 16 aa, synthetic construct
CALGGENAGNNRKLIW

SEQ ID NO: 20 - protein, 15 aa, synthetic construct
CAVSEIYNQGGKLIF

SEQ ID NO: 21 - protein, 15 aa, synthetic construct
CAFADSTSGTYKYIF

SEQ ID NO: 22 - protein, 14 aa, synthetic construct
CAYEAGGANSKLTF

SEQ ID NO: 23 - protein, 14 aa, synthetic construct
CATDPSNQGGKLIF

SEQ ID NO: 24 - protein, 15 aa, synthetic construct
CAVSENSGGYQKVTF

SEQ ID NO: 25 - protein, 15 aa, synthetic construct
CALIEFSGGYNKLIF

SEQ ID NO: 26 - protein, 15 aa, synthetic construct
CLVGDLNTGFQKLVF

SEQ ID NO: 27 - protein, 14 aa, synthetic construct
CATDLIQGAQKLVF

SEQ ID NO: 28 - protein, 15 aa, synthetic construct
CAMSEYGGATNKLIF

SEQ ID NO: 29 - protein, 14 aa, synthetic construct
CAESHSGSARQLTF

SEQ ID NO: 30 - protein, 14 aa, synthetic construct
CGTELTSGTYKYIF

SEQ ID NO: 31 - protein, 15 aa, synthetic construct
CLVGDGSGNTGKLIF

SEQ ID NO: 32 - protein, 14 aa, synthetic construct
CATDYGGATNKLIF

SEQ ID NO: 33 - protein, 15 aa, synthetic construct
CVVSDGSGGYNKLIF

SEQ ID NO: 34 - protein, 13 aa, synthetic construct
CAELLSGTYKYIF

SEQ ID NO: 35 - protein, 15 aa, synthetic construct
CAGQDSGGSNYKLTF

SEQ ID NO: 36 - protein, 15 aa, synthetic construct
CAVSDVTGGGNKLTF

SEQ ID NO: 37 - protein, 14 aa, synthetic construct
CAVEGGTSYGKLTF

SEQ ID NO: 38 - protein, 13 aa, synthetic construct
CSDALQGAQKLVF

SEQ ID NO: 39 - protein, 13 aa, synthetic construct
CAEGSSNTGKLIF

SEQ ID NO: 40 - protein, 15 aa, synthetic construct
CCAPDGSSNTGKLIF

SEQ ID NO: 41 - protein, 15 aa, synthetic construct
CALSDGTGGGNKLTF

SEQ ID NO: 42 - protein, 16 aa, synthetic construct
CAFMTQWGGSQGNLIF

SEQ ID NO: 43 - protein, 15 aa, synthetic construct
CCGGGGGGSQGNLIF

SEQ ID NO: 44 - protein, 15 aa, synthetic construct
CAGLNYGGSQGNLIF

SEQ ID NO: 45 - protein, 16 aa, synthetic construct
CAASAFSSGSARQLTF

SEQ ID NO: 46 - protein, 15 aa, synthetic construct
CAVNPHGSAGNMLTF

SEQ ID NO: 47 - protein, 15 aa, synthetic construct
CAVGNYGGSQGNLIF

SEQ ID NO: 48 - protein, 60 aa, synthetic construct
MAEPRQEFEVMEDHAGTYGLGDRKDQGGYTMHQDQEGDTDAGLKESPLQTPTEDGSEEPG

SEQ ID NO: 49 - protein, 60 aa, synthetic construct
SETSDAKSTPTAEAEEAGIGDTPSLEDEAAGHVTQARMVSKSKDGTGSDDKKAKGADGKT

SEQ ID NO: 50 - protein, 60 aa, synthetic construct
KIATPRGAAPPGQKGQANATRIPAKTPPAPKTPPSSGEPPKSGDRSGYSSPGSPGTPGSR

SEQ ID NO: 51 - protein, 60 aa, synthetic construct
SRTPSLPTPPTREPKKVAVVRTPPKSPSSAKSRLQTAPVPMPDLKNVKSKIGSTENLKHQ

SEQ ID NO: 52 - protein, 60 aa, synthetic construct
PGGGKVQIINKKLDLSNVQSKCGSKDNIKHVPGGGSVQIVYKPVDLSKVTSKCGSLGNIH

SEQ ID NO: 53 - protein, 60 aa, synthetic construct
HKPGGGQVEVKSEKLDFKDRVQSKIGSLDNITHVPGGGNKKIETHKLTFRENAKAKTDHG

SEQ ID NO: 54 - protein, 52 aa, synthetic construct
AEIVYKSPVVSGDTSPRHLSNVSSTGSIDMVDSPQLATLADEVSASLAKQGL

(19) United States
(12) Patent Application Publication
(10) Pub. No.: US 2023/0340587 A1
(43) Pub. Date: Oct. 26, 2023

(54) AMINO ACID COMPLEMENTARITY SCORING AND USES THEREOF

(71) Applicant: University of South Florida, Tampa, FL (US)

(72) Inventors: George Blanck, Tampa, FL (US); Boris Il'ich Chobrutskiy, Portland, OR (US); Taha Ibrahim Huda, Belleair, FL (US); Andrea Diviney Chobrutskiy, Portland, OR (US); Michael Joseph Diaz, Tampa, FL (US); Etienne Gozlan, Palm Beach Gardens, FL (US)

(21) Appl. No.: 18/174,164

(22) Filed: Feb. 24, 2023

Related U.S. Application Data
(60) Provisional application No. 63/268,574, filed on Feb. 25, 2022.

(51) Int. Cl. C12Q 1/6869; C12Q 1/6883

(52) U.S. Cl. CPC C12Q 1/6869; C12Q 1/6883; C12Q 2600/112; C12Q 2600/118; C12Q 2600/156

(57) ABSTRACT

The present disclosure relates to methods of detecting, treating, and/or preventing the progression of diseases, such as Alzheimer's disease using a complementarity scoring method.

### CROSS REFERENCE TO RELATED APPLICATIONS

This application claims the benefit of U.S. Provisional Patent Application Ser. No. 63/268,574 filed Feb. 25, 2022, which is expressly incorporated herein by reference in its entirety.

### REFERENCE TO SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on May 16, 2023, is entitled “11001-155US1.xml”, and is 48,888 bytes in size.

### FIELD

The present disclosure relates to methods of detecting, treating, and/or preventing the progression of diseases, such as Alzheimer's disease using a complementarity scoring method.

### BACKGROUND

Chemical complementarity is a key feature of scoring protein-protein interactions. Specific protein-protein interactions are accomplished via molecular recognition between distinct amino acids sequences with a polypeptide or protein. Thus, different polypeptides or proteins with complementary amino acids can interact, and in some cases directly bind to each other. Furthermore, this interaction can be examined and quantified by scoring the complementarity between amino acids. This approach allows for detecting immune responses to disease progression by assessing immune receptor interactions with disease associated proteins. There has been limited advancement in the understanding of immune system responses and developing effective therapeutics in many diseases, including neurodegenerative diseases such as Alzheimer's disease (AD). Thus given limitations of detecting and treating diseases, such as AD, there is need to address the aforementioned problems mentioned above by developing methods of detecting disease progression prior to onset of late or advanced stages of disease. The methods disclosed herein address these and other needs.

### SUMMARY

The present disclosure also provides methods detecting, treating, and/or preventing progression of Alzheimer's disease using an amino acid complementarity scoring.

In one aspect, disclosed herein is a method of detecting a progression of Alzheimer's disease in a subject, comprising obtaining and isolating a nucleic acid sample from a biological sample derived from the subject, sequencing a first nucleic acid sequence of a complementary determining region (CDR) of an antigen-binding protein and sequencing a second nucleic acid sequence of a misfolded or aggregated protein, or a fragment thereof, associated with Alzheimer's disease, translating the first nucleic acid sequence into an amino acid sequence of the complementary determining region (CDR) of the antigen-binding protein and translating the second nucleic acid sequence into an amino acid sequence of the misfolded or aggregated protein, or a fragment thereof, associated with Alzheimer's disease, determining a chemical complementarity score between the amino acid sequence from the CDR and the amino acid sequence of the protein associated with Alzheimer's disease, and detecting the progression of Alzheimer's disease in the subject, wherein a first chemical complementarity score is higher in a first biological sample derived from the subject compared to a chemical complementarity score from a reference control, and wherein a second chemical complementarity score is higher in a second biological sample derived from the subject compared the first chemical complementarity score and the chemical complementarity score from the reference control.

In some embodiments, the chemical complementarity score represents an interaction between one or more amino acids of the CDR of the antigen-binding protein and one or more amino acids of the protein associated with Alzheimer's disease.

In some embodiments, a low chemical complementarity score indicates a low-level (transentorhinal) Braak stage of Alzheimer's disease. In some embodiments, a high chemical complementarity score indicates a mid-level (limbic stage) or an advanced level (neocortical) Braak stage of Alzheimer's disease.

In some embodiments, the low-level Braak stage comprises Braak stage I or Braak stage II. In some embodiments, the mid-level Braak stage comprises Braak stage III or Braak stage IV, and wherein the advanced-level Braak stage comprises Braak stage V or Braak stage VI.

In some embodiments, the antigen-binding protein comprises a T cell receptor alpha (TRA) chain or a T cell receptor beta (TRB) chain.

In some embodiments, the misfolded or aggregated protein associated with Alzheimer's disease comprises a tau protein, a fragment thereof, or a related protein.

In some embodiments, the method comprises administering a therapeutic composition after detecting the progression of Alzheimer's disease in the subject, wherein the therapeutic composition comprises donepezil, galantamine, rivastigmine, memantine, lecanemab, aducanumab, or a related composition.

In some embodiments, the biological sample comprises a blood sample.

In some embodiments, the subject is a human.

In one aspect, disclosed herein is a method of treating or preventing a progression of Alzheimer's disease in a subject in need thereof, the method comprising obtaining and isolating a nucleic acid sample from a biological sample derived from the subject, sequencing a first nucleic acid sequence of a complementary determining region (CDR) of an antigen-binding protein and sequencing a second nucleic acid sequence of a misfolded or aggregated protein, or a fragment thereof, associated with Alzheimer's disease, translating the first nucleic acid sequence into an amino acid sequence of the complementary determining region (CDR) of the antigen-binding protein and translating the second nucleic acid sequence into an amino acid sequence of the misfolded or aggregated protein, or a fragment thereof, associated with Alzheimer's disease, determining a chemical complementarity score between the amino acid sequence from the CDR and the amino acid sequence of the protein associated with Alzheimer's disease, detecting the progression of Alzheimer's disease in the subject, wherein a first chemical complementarity score is higher in a first biological sample derived from the subject compared to a chemical complementarity score from a reference control, and wherein a second chemical complementarity score is higher in a second biological sample derived from the subject compared the first chemical complementarity score and the chemical complementarity score from the reference control, and administering a therapeutic composition to slow the progression of Alzheimer's disease in the subject.

In some embodiments, the chemical complementarity score represents an interaction between one or more amino acids of the CDR of the antigen-binding protein and one or more amino acids of the protein associated with Alzheimer's disease.

In some embodiments, a low chemical complementarity score indicates a low-level (transentorhinal) Braak stage of Alzheimer's disease. In some embodiments, a high chemical complementarity score indicates a mid-level (limbic stage) or an advanced level (neocortical) Braak stage of Alzheimer's disease.

In some embodiments, the low-level Braak stage comprises Braak stage I or Braak stage II. In some embodiments, the mid-level Braak stage comprises Braak stage III or Braak stage IV, and wherein the advanced-level Braak stage comprises Braak stage V or Braak stage VI.

In some embodiments, the antigen-binding protein comprises a T cell receptor (TCR) alpha (TRA) chain or a TCR beta (TRB) chain.

In some embodiments, the misfolded or aggregated protein associated with Alzheimer's disease comprises a tau protein, a fragment thereof, or a related protein.

In some embodiments, the therapeutic composition comprises donepezil, galantamine, rivastigmine, memantine, lecanemab, aducanumab, or a related composition.

### BRIEF DESCRIPTION OF FIGURES

The accompanying figures, which are incorporated in and constitute a part of this specification, illustrate several aspects described below.

FIG. 1 shows the violin plot correlating higher Braak stage (worse clinical outcome) with higher isoelectric points in brain-derived TRA CDR3s. (Kruskal-Wallis p-value: 0.044).

FIG. 2 shows the Alzheimer's disease (AD) clinical progression and Braak Stages.

FIG. 3 shows a diagram of the immune system in AD.

FIG. 4 shows the dual histogram correlating higher Braak stage with higher hydropathy complementarity score for blood TRA CDR3s and Tau segment 5 (p=0.017).

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, e.g., prevention of a disorder or other undesirable physiological condition. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “patient” is any subject receiving or awaiting to receive medical care or treatment. A “patient” can be a human, non-human primate, non-human mammal, or any other vertebrate or non-vertebrate animal. For example, a patient can be a human, a dog, a cat, a monkey, an ape, a bird, a frog, a mouse, a rabbit, a fish, a jellyfish, or snake.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating or impeding one or more causes of a disorder or condition. Treatments according to the disclosure may be applied preventively, prophylactically, palliatively, or remedially. Treatments are administered to a subject prior to onset (e.g., before obvious signs of a neurodegenerative disease, such as Alzheimer's disease), during early onset (e.g., upon initial signs and symptoms of a neurodegenerative disease, such as Alzheimer's disease), or after an established development of a neurodegenerative disease, such as Alzheimer's disease.

References are made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110). A “protein”, “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like. The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

“Fragments” used herein refer to a reference amino acid sequence or nucleotide sequence. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

The term “administer”, “administering”, or derivatives thereof refer to delivering a composition to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “T cell” refers to a type of lymphocyte that is one of the most important white blood cells of the immune system. T cells can be distinguished from other lymphocytes by the presence of a T-cell receptor (TCR) on their cell surface. The immune-mediated cell death function of T cells is carried by two major subtypes: CD8⁺ “killer” T cells and CD4⁺ “helper T cells.

A “control” or “reference control” is an alternative subject or sample used in an experiment for comparison purpose. A control can be “positive” or “negative.” For example, where the purpose of the experiment is to determine a correlation of an altered expression level of a receptor, protein, or fragments thereof, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

A “gene product” refers to the amino acid (e.g., peptide or polypeptide) generated when a gene is transcribed and translated.

As used herein, the terms “translate”, “translated”, and “translating” refers to the process in which cellular organelles, called ribosomes, in the cytoplasm or endoplasmic reticulum (ER) synthesize proteins after process of transcription of DNA to RNA in a cell's nucleus. This process is also a component of the process of gene expression. Specifically, messenger RNA (mRNA) is decoded in a ribosome, outside the nucleus, to produce a specific amino acid chain, or polypeptide. The polypeptide later folds into an active/functional protein in the cell. The ribosome facilitates decoding by inducing the binding of complementary transfer RNA (tRNA) anticodon sequences to mRNA codons. The tRNAs carry specific amino acids that are chained together into a polypeptide as the mRNA passes through and is “read” by the ribosome. The entire process includes 3 phases: 1) Initiation, 2) Elongation, and 3) Termination.

The term “interaction” refers to an action that occurs as two or more objects have an effect on one another either with or without physical contact. In terms of biological interactions, cell, proteins, and other macromolecules can have said effects on one another to impact biological functions, such as cell growth, cell death, and cell signaling pathways.

As used herein, a “progressive disease” refers to a disease or physical ailment whose course in most cases is the worsening, growth, or spread of disease. This worsening will continue until serious debility, organ failure, or death occurs. Some progressive diseases can be halted and/or reversed by treatment (surgical, dietary, or lifestyle interventions). Some examples of progressive diseases include, but are not limited to various cancers, Alzheimer's disease, Huntington's disease, multiple sclerosis, and Parkinson's disease.

A “neurodegenerative disease” is caused by the progressive loss of structure or function of neurons or glial cells, which make up the nervous system. These diseases include but are not limited to amyotrophic lateral sclerosis (ALS), multiple sclerosis, Parkinson's disease, Alzheimer's disease, Huntington's disease, and prion diseases. Neurodegenerative diseases can lead to cognitive and physical impairments, neuroinflammation (inflammation of the brain and spinal cord), and deterioration of brain and spinal cord tissues.

The “blood-brain barrier” or the “BBB” refers to the highly selective semipermeable border of endothelial cells that prevents certain small molecules circulating in the blood from crossing into the extracellular fluid of the central nervous system.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

### Methods of Detecting, Treating, and/or Preventing Progression of Alzheimer's Disease

Alzheimer's disease represents one of many neurodegenerative diseases, progressive diseases, and is a common type of dementia resulting in memory loss and inability to communicate and respond to the surround environment. Specifically, Alzheimer's disease affects areas of the brain controlling thoughts, memories, and speech/language. Alzheimer's disease also worsens over time, wherein symptoms gradually worsen over a number of years ranging from 2 years to 20 years. To date, there are no effective methods of tracking Alzheimer's disease progression or determining if a subject will progress relatively quickly or slowly. Therefore, the present disclosure addresses this need providing methods detecting, treating, and/or preventing progression of Alzheimer's disease using an amino acid complementarity scoring.

In one aspect, disclosed herein is a method of detecting a progression of Alzheimer's disease in a subject, comprising obtaining and isolating a nucleic acid sample from a biological sample derived from the subject, sequencing a first nucleic acid sequence of a complementary determining region (CDR) of an antigen-binding protein and sequencing a second nucleic acid sequence of a misfolded or aggregated protein, or a fragment thereof, associated with Alzheimer's disease, translating the first nucleic acid sequence into an amino acid sequence of the complementary determining region (CDR) of the antigen-binding protein and translating the second nucleic acid sequence into an amino acid sequence of the misfolded or aggregated protein, or a fragment thereof, associated with Alzheimer's disease, determining a chemical complementarity score between the amino acid sequence from the CDR and the amino acid sequence of the protein associated with Alzheimer's disease, and detecting the progression of Alzheimer's disease in the subject, wherein a first chemical complementarity score is higher in a first biological sample derived from the subject compared to a chemical complementarity score from a reference control, and wherein a second chemical complementarity score is higher in a second biological sample derived from the subject compared the first chemical complementarity score and the chemical complementarity score from the reference control.

Stages of Alzheimer's disease and other neurodegenerative diseases, such as Parkinson's disease, can be classified using the Braak staging methods to identify to degree of disease pathology and progression. Specifically, in Alzheimer's disease, the Braak staging method comprises 6 stages comprising: Stages I and II wherein neurofibrillary tangles of misfolded/aggregated proteins are present in the transenterorhinal region of the brain; Stages III and IV wherein neurofibrillary tangles of misfolded/aggregated proteins progress into the limbic region of the brain; and Stages V and VI wherein neurofibrillary tangles of misfolded/aggregated proteins progress into the neocortical regions of the brain.

As used herein, a “CDR” refers to an immunoglobulin (Ig) region within an antigen-binding protein or an antibody comprising a variable region that determines specific antibody binding to an antigen protein, peptide, chemical, compound, or a biological composition.

As used herein, an “antigen-binding protein” refers to any protein, recombinant protein, recombinant polypeptides, or antibodies composed of an antibody variable light-chain amino acid sequence (V_{L}) tethered to a variable heavy-chain sequence (V_{H}) by a designated peptide that links the carboxyl terminus of the V_{L} sequence to the amino terminus of the V_{H} sequence. An antigen binding protein also refers to a protein capable of recognizing and binding an antigen or target molecule for the purpose of initiating an immune response.

It should be noted that after translation of polypeptide chains, said polypeptides can be processed and folded to become biologically active proteins into its native three dimensional structure. Proper folding of a protein is crucial to its biological functions. In contrast, improper folding can lead to accumulation and aggregation of dysfunctional proteins leading to development and progression of diseases. As used herein, “aggregation of proteins” refers to an abnormal association, interaction, and/or binding of proteins, usually misfolded proteins, into larger grouping or clump of protein structures which tend to be insoluble and contributes to disease.

In some embodiments, the chemical complementarity score represents an interaction between one or more amino acids of the CDR of the antigen-binding protein and one or more amino acids of the protein associated with Alzheimer's disease.

In some embodiments, the chemical complementarity score is a direct correlation between the interaction or binding of one or more amino acids of the CDR of the antigen-binding protein and one or more amino acids of the protein associated with Alzheimer's disease.

It should be understood that there can be numerous CDR3 amino acid sequences within a biological sample. There can also be numerous CDR3 amino acid sequences within a blood sample. In some embodiments, the CDR3 amino acid sequence comprises a repetitive amino acid sequence.

In some embodiments, a low chemical complementarity score indicates a low-level (transentorhinal) Braak stage of Alzheimer's disease. In some embodiments, a high chemical complementarity score indicates a mid-level (limbic stage) or an advanced level (neocortical) Braak stage of Alzheimer's disease.

In some embodiments, a low chemical complementarity score indicates Braak Stage I or Braak Stage I or Braak Stage II. In some embodiments, a low chemical complementarity score indicates Braak Stage I. In some embodiments, a low chemical complementarity score indicates Braak Stage II.

In some embodiments, a high chemical complementarity score indicates Braak Stage III, IV, V, or VI. In some embodiments, a high chemical complementarity score indicates Braak Stage III. In some embodiments, a high chemical complementarity score indicates Braak Stage IV. In some embodiments, a high chemical complementarity score indicates Braak Stage V. In some embodiments, a high chemical complementarity score indicates Braak Stage VI.

In some embodiments, the low-level Braak stage comprises Braak stage I or Braak stage II. In some embodiments, the mid-level Braak stage comprises Braak stage III or Braak stage IV, and wherein the advanced-level Braak stage comprises Braak stage V or Braak stage VI.

In some embodiments, the antigen-binding protein comprises a T cell receptor alpha (TRA) chain or a T cell receptor beta (TRB) chain. In some embodiments, the antigen-binding protein comprises a TRA chain. In some embodiments, the antigen-binding protein comprises a TRB chain.

In some embodiments, the antigen-binding protein comprises a B cell receptor. In some embodiments, the antigen-binding protein comprises a heavy chain B cell receptor. In some embodiments, the antigen-binding protein comprises an antibody.

In some embodiments, the misfolded or aggregated protein associated with Alzheimer's disease comprises a tau protein, a fragment thereof, or a related protein. In some embodiments, the misfolded or aggregated protein associated with Alzheimer's disease comprises amyloid-beta peptide, amyloid precursor peptide (APP), alpha-synuclein, TAR DNA-binding protein 43 (TDP-43), or prion proteins.

In some embodiments, the method comprises administering a therapeutic composition after detecting the progression of Alzheimer's disease in the subject, wherein the therapeutic composition comprises donepezil, galantamine, rivastigmine, memantine, lecanemab, aducanumab, or a related composition.

In some embodiments, the biological sample comprises a blood sample. In some embodiments, the biological sample comprises a brain tissue. In some embodiments, the biological sample is a spinal cord tissue or fluid. In some embodiments, the biological sample is a cerebrospinal fluid (CSF).

In some embodiments, the subject is a human.

In one aspect, disclosed herein is a method of treating or preventing a progression of Alzheimer's disease in a subject in need thereof, the method comprising obtaining and isolating a nucleic acid sample from a biological sample derived from the subject, sequencing a first nucleic acid sequence of a complementary determining region (CDR) of an antigen-binding protein and sequencing a second nucleic acid sequence of a misfolded or aggregated protein, or a fragment thereof, associated with Alzheimer's disease, translating the first nucleic acid sequence into an amino acid sequence of the complementary determining region (CDR) of the antigen-binding protein and translating the second nucleic acid sequence into an amino acid sequence of the misfolded or aggregated protein, or a fragment thereof, associated with Alzheimer's disease, determining a chemical complementarity score between the amino acid sequence from the CDR and the amino acid sequence of the protein associated with Alzheimer's disease, detecting the progression of Alzheimer's disease in the subject, wherein a first chemical complementarity score is higher in a first biological sample derived from the subject compared to a chemical complementarity score from a reference control, and wherein a second chemical complementarity score is higher in a second biological sample derived from the subject compared the first chemical complementarity score and the chemical complementarity score from the reference control, and administering a therapeutic composition to slow the progression of Alzheimer's disease in the subject.

In some embodiments, the chemical complementarity score represents an interaction between one or more amino acids of the CDR of the antigen-binding protein and one or more amino acids of the protein associated with Alzheimer's disease.

In some embodiments, the chemical complementarity score is a direct correlation between the interaction or binding of one or more amino acids of the CDR of the antigen-binding protein and one or more amino acids of the protein associated with Alzheimer's disease.

In some embodiments, a low chemical complementarity score indicates a low-level (transentorhinal) Braak stage of Alzheimer's disease. In some embodiments, a high chemical complementarity score indicates a mid-level (limbic stage) or an advanced level (neocortical) Braak stage of Alzheimer's disease.

In some embodiments, a low chemical complementarity score indicates Braak Stage I or Braak Stage I or Braak Stage II. In some embodiments, a low chemical complementarity score indicates Braak Stage I. In some embodiments, a low chemical complementarity score indicates Braak Stage II.

In some embodiments, a high chemical complementarity score indicates Braak Stage III, IV, V, or VI. In some embodiments, a high chemical complementarity score indicates Braak Stage III. In some embodiments, a high chemical complementarity score indicates Braak Stage IV. In some embodiments, a high chemical complementarity score indicates Braak Stage V. In some embodiments, a high chemical complementarity score indicates Braak Stage VI.

In some embodiments, the low-level Braak stage comprises Braak stage I or Braak stage II. In some embodiments, the mid-level Braak stage comprises Braak stage III or Braak stage IV, and wherein the advanced-level Braak stage comprises Braak stage V or Braak stage VI.

In some embodiments, the antigen-binding protein comprises a T cell receptor alpha (TRA) chain or a T cell receptor beta (TRB) chain. In some embodiments, the antigen-binding protein comprises a TRA chain. In some embodiments, the antigen-binding protein comprises a TRB chain.

In some embodiments, the antigen-binding protein comprises a B cell receptor. In some embodiments, the antigen-binding protein comprises a heavy chain B cell receptor. In some embodiments, the antigen-binding protein comprises an antibody.

In some embodiments, the misfolded or aggregated protein associated with Alzheimer's disease comprises a tau protein, a fragment thereof, or a related protein. In some embodiments, the misfolded or aggregated protein associated with Alzheimer's disease comprises amyloid-beta peptide, amyloid precursor peptide (APP), alpha-synuclein, TAR DNA-binding protein 43 (TDP-43), or prion proteins.

In some embodiments, the therapeutic composition comprises donepezil, galantamine, rivastigmine, memantine, lecanemab, aducanumab, or a related composition.

In some embodiments, the biological sample comprises a blood sample. In some embodiments, the biological sample comprises a brain tissue. In some embodiments, the biological sample is a spinal cord tissue or fluid. In some embodiments, the biological sample is a cerebrospinal fluid (CSF).

In some embodiments, the subject is a human.

### Methods of Detecting, Treating, and/or Preventing Progression of a Neurodegenerative Disease

Neurodegenerative diseases represent a group of disease in which cells of the central nervous system stop functioning or die. Individuals with any one neurodegenerative disease usually presents with symptoms that worsen over time and does not have a cure. Most neurodegenerative diseases, such as Alzheimer's disease and Parkinson's disease, comprise a classification system, term the Braak Stages, which describe the degree of pathology or disease progression.

For example, the Braak stage of Parkinson's disease comprises: Stage I wherein Lewy neurites, a thread-like alpha-synuclein aggregates, are present in the lower brainstem and the olfactory system of the brain; Stage II wherein Lewy neurites are progress into the medulla oblongata; Stage III wherein Lewy neurites progress into the substantia nigra and Lewy bodies, rounded alpha-synuclein aggregates, begin to form in the pars compacta; Stage IV wherein pathology progresses into the amygdala and thalamus, and dopaminergic neurons become destructive and dysfunctional; Stage V wherein disease pathology begin progressing into the neocortex of the brain, and spreads further into the temporal, parietal, and frontal lobes of the brain; and Stage VI wherein the disease pathology has fully invaded into the neocortex.

In one aspect, disclosed herein is a method of treating or preventing a progression of a neurodegenerative disease in a subject in need thereof, the method comprising obtaining and isolating a nucleic acid sample from a biological sample derived from the subject, sequencing a first nucleic acid sequence of a complementary determining region (CDR) of an antigen-binding protein and sequencing a second nucleic acid sequence of a misfolded or aggregated protein, or a fragment thereof, associated with the neurodegenerative disease, translating the first nucleic acid sequence into an amino acid sequence of the complementary determining region (CDR) of the antigen-binding protein and translating the second nucleic acid sequence into an amino acid sequence of the misfolded or aggregated protein, or a fragment thereof, associated with the neurodegenerative disease, determining a chemical complementarity score between the amino acid sequence from the CDR and the amino acid sequence of the protein associated with the neurodegenerative disease, and detecting the progression of the neurodegenerative disease in the subject, wherein a first chemical complementarity score is higher in a first biological sample derived from the subject compared to a chemical complementarity score from a reference control, and wherein a second chemical complementarity score is higher in a second biological sample derived from the subject compared the first chemical complementarity score and the chemical complementarity score from the reference control.

In some embodiments, the neurodegenerative disease includes, but is not limited to Alzheimer's disease, ataxia, Huntington's disease, Parkinson's disease, amyotrophic lateral sclerosis (ALS), Friedreich ataxia, Lewy body disease, spinal muscular atrophy, Alpers' disease, Batten disease, Cerebro-oculo-facio-skeletal syndrome, Leigh syndrome, Prion diseases, monomelic amyotrophy, multiple system atrophy, striatonigral degeneration, motor neuron disease, multiple sclerosis (MS), Creutzfeldt-Jakob disease, Parkinsonism, spinocerebellar ataxia, dementia, and other related diseases.

In some embodiments, the chemical complementarity score represents an interaction between one or more amino acids of the CDR of the antigen-binding protein and one or more amino acids of the protein associated with the neurodegenerative disease.

In some embodiments, the chemical complementarity score is a direct correlation between the interaction or binding of one or more amino acids of the CDR of the antigen-binding protein and one or more amino acids of the protein associated with the neurodegenerative disease.

It should be understood that there can be numerous CDR3 amino acid sequences within a biological sample. There can also be numerous CDR3 amino acid sequences within a blood sample. In some embodiments, the CDR3 amino acid sequence comprises a repetitive amino acid sequence.

In some embodiments, a low chemical complementarity score indicates a low-level (transentorhinal) Braak stage of a neurodegenerative disease. In some embodiments, a high chemical complementarity score indicates a mid-level (limbic stage) or an advanced level (neocortical) Braak stage of a neurodegenerative disease.

In some embodiments, a low chemical complementarity score indicates Braak Stage I or Braak Stage I or Braak Stage II. In some embodiments, a low chemical complementarity score indicates Braak Stage I. In some embodiments, a low chemical complementarity score indicates Braak Stage II.

In some embodiments, a high chemical complementarity score indicates Braak Stage III, IV, V, or VI. In some embodiments, a high chemical complementarity score indicates Braak Stage III. In some embodiments, a high chemical complementarity score indicates Braak Stage IV. In some embodiments, a high chemical complementarity score indicates Braak Stage V. In some embodiments, a high chemical complementarity score indicates Braak Stage VI.

In some embodiments, the low-level Braak stage comprises Braak stage I or Braak stage II. In some embodiments, the mid-level Braak stage comprises Braak stage III or Braak stage IV, and wherein the advanced-level Braak stage comprises Braak stage V or Braak stage VI.

In some embodiments, the antigen-binding protein comprises a T cell receptor alpha (TRA) chain or a T cell receptor beta (TRB) chain. In some embodiments, the antigen-binding protein comprises a TRA chain. In some embodiments, the antigen-binding protein comprises a TRB chain.

In some embodiments, the antigen-binding protein comprises a B cell receptor. In some embodiments, the antigen-binding protein comprises a heavy chain B cell receptor. In some embodiments, the antigen-binding protein comprises an antibody.

In some embodiments, the misfolded or aggregated protein associated with the neurodegenerative disease comprises a tau protein, a fragment thereof, or a related protein. In some embodiments, the misfolded or aggregated protein associated with the neurodegenerative disease comprises amyloid-beta peptide, amyloid precursor peptide (APP), alpha-synuclein, TAR DNA-binding protein 43 (TDP-43), or prion proteins.

In some embodiments, the therapeutic composition comprises donepezil, galantamine, rivastigmine, memantine, lecanemab, aducanumab, amantadine, apomorphine, benzatropine, biperiden, bromocriptine, carbidopa, entacapone, levodopa, pramipexole, procyclidine, rasagiline, ropinirole, rotigotine, selegiline, tolcapone, trihexyphenidyl, azathioprine, cyclophosphamide, dexamethasone, fingolimod, glatiramer, dalfampridine, interferon 1a, interferon 1b, natalizumab, prednisone, riluzole, or a related compositions.

In some embodiments, the biological sample comprises a blood sample. In some embodiments, the biological sample comprises a brain tissue. In some embodiments, the biological sample is a spinal cord tissue or fluid. In some embodiments, the biological sample is a cerebrospinal fluid (CSF).

In some embodiments, the subject is a human.

In one aspect, disclosed herein is a method of treating or preventing a progression of a neurodegenerative disease in a subject in need thereof, the method comprising obtaining and isolating a nucleic acid sample from a biological sample derived from the subject, sequencing a first nucleic acid sequence of a complementary determining region (CDR) of an antigen-binding protein and sequencing a second nucleic acid sequence of a misfolded or aggregated protein, or a fragment thereof, associated with the neurodegenerative disease, translating the first nucleic acid sequence into an amino acid sequence of the complementary determining region (CDR) of the antigen-binding protein and translating the second nucleic acid sequence into an amino acid sequence of the misfolded or aggregated protein, or a fragment thereof, associated with the neurodegenerative disease, determining a chemical complementarity score between the amino acid sequence from the CDR and the amino acid sequence of the protein associated with the neurodegenerative disease, detecting the progression of the neurodegenerative disease in the subject, wherein a first chemical complementarity score is higher in a first biological sample derived from the subject compared to a chemical complementarity score from a reference control, and wherein a second chemical complementarity score is higher in a second biological sample derived from the subject compared the first chemical complementarity score and the chemical complementarity score from the reference control, and administering a therapeutic composition to slow the progression of the neurodegenerative disease in the subject.

In some embodiments, the neurodegenerative disease includes, but is not limited to Alzheimer's disease, ataxia, Huntington's disease, Parkinson's disease, amyotrophic lateral sclerosis (ALS), Friedreich ataxia, Lewy body disease, spinal muscular atrophy, Alpers' disease, Batten disease, Cerebro-oculo-facio-skeletal syndrome, Leigh syndrome, Prion diseases, monomelic amyotrophy, multiple system atrophy, striatonigral degeneration, motor neuron disease, multiple sclerosis (MS), Creutzfeldt-Jakob disease, Parkinsonism, spinocerebellar ataxia, dementia, and other related diseases.

In some embodiments, the chemical complementarity score represents an interaction between one or more amino acids of the CDR of the antigen-binding protein and one or more amino acids of the protein associated with the neurodegenerative disease.

In some embodiments, the chemical complementarity score is a direct correlation between the interaction or binding of one or more amino acids of the CDR of the antigen-binding protein and one or more amino acids of the protein associated with the neurodegenerative disease.

In some embodiments, a low chemical complementarity score indicates a low-level (transentorhinal) Braak stage of a neurodegenerative disease. In some embodiments, a high chemical complementarity score indicates a mid-level (limbic stage) or an advanced level (neocortical) Braak stage of a neurodegenerative disease.

In some embodiments, a low chemical complementarity score indicates Braak Stage I or Braak Stage I or Braak Stage II. In some embodiments, a low chemical complementarity score indicates Braak Stage I. In some embodiments, a low chemical complementarity score indicates Braak Stage II.

In some embodiments, a high chemical complementarity score indicates Braak Stage III, IV, V, or VI. In some embodiments, a high chemical complementarity score indicates Braak Stage III. In some embodiments, a high chemical complementarity score indicates Braak Stage IV. In some embodiments, a high chemical complementarity score indicates Braak Stage V. In some embodiments, a high chemical complementarity score indicates Braak Stage VI.

In some embodiments, the low-level Braak stage comprises Braak stage I or Braak stage II. In some embodiments, the mid-level Braak stage comprises Braak stage III or Braak stage IV, and wherein the advanced-level Braak stage comprises Braak stage V or Braak stage VI.

In some embodiments, the antigen-binding protein comprises a T cell receptor alpha (TRA) chain or a T cell receptor beta (TRB) chain. In some embodiments, the antigen-binding protein comprises a TRA chain. In some embodiments, the antigen-binding protein comprises a TRB chain.

In some embodiments, the antigen-binding protein comprises a B cell receptor. In some embodiments, the antigen-binding protein comprises a heavy chain B cell receptor. In some embodiments, the antigen-binding protein comprises an antibody.

In some embodiments, the misfolded or aggregated protein associated with the neurodegenerative disease comprises a tau protein, a fragment thereof, or a related protein. In some embodiments, the misfolded or aggregated protein associated with the neurodegenerative disease comprises amyloid-beta peptide, amyloid precursor peptide (APP), alpha-synuclein, TAR DNA-binding protein 43 (TDP-43), or prion proteins.

In some embodiments, the therapeutic composition comprises donepezil, galantamine, rivastigmine, memantine, lecanemab, aducanumab, amantadine, apomorphine, benzatropine, biperiden, bromocriptine, carbidopa, entacapone, levodopa, pramipexole, procyclidine, rasagiline, ropinirole, rotigotine, selegiline, tolcapone, trihexyphenidyl, azathioprine, cyclophosphamide, dexamethasone, fingolimod, glatiramer, dalfampridine, interferon 1a, interferon 1b, natalizumab, prednisone, riluzole, or a related compositions.

In some embodiments, the biological sample comprises a blood sample. In some embodiments, the biological sample comprises a brain tissue. In some embodiments, the biological sample is a spinal cord tissue or fluid. In some embodiments, the biological sample is a cerebrospinal fluid (CSF).

In some embodiments, the subject is a human.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Immunogenomics Parameters for Patient Stratification in Alzheimer's Disease

Despite the fact that only modest adaptive immune system related approaches to treating Alzheimer's disease (AD) are available, an immunogenomics approach to the study of Alzheimer's disease has not yet substantially advanced. Thus, T-cell receptor alpha (TRA) complementarity determining region-3 (CDR3) physicochemical features were characterized. TRA CDR3 homology groups were then identified and represented by TRA recombination reads extracted from 2665 AD-related, blood- and brain-derived exome files. A higher isoelectric value for the brain TRA CDR3s was found to be associated with a higher (clinically worse) Braak stage and that a number of TRA CDR3 chemical homology groups, in particular representing bloodborne TRA CDR3s, were associated with higher or lower Braak stages. Lastly, greater chemical complementarity of both blood- and brain-derived TRA CDR3s and Tau, based on a described CDR3-candidate antigen chemical complementarity scoring process (adaptivematch.com), was associated with higher Braak stages.

Alzheimer's disease (AD) is an irreversible neurodegenerative disease where the pathophysiology is still not fully understood. However, in all patients, the processing of amyloid precursor protein (APP) is altered, leading to deposition and aggregation of β-amyloid, in turn associated with hyperphosphorylation of Tau (MAPT) and neurofibrillary tangles. Keeping in mind this pathophysiology, additional contributions from other patient features, including genetics, the environment, and the immune system, have been assessed.

In particular, it has been identified that low levels of chronic inflammation in aging, known as “inflammaging”, is a risk factor for decreased brain function and AD. This neuroinflammation is thought to exacerbate AD via the brain's innate immune cells, including the microglia and macrophages. Furthermore, a role of the adaptive immune response has been partly elucidated, as it has been shown that gamma T-cell receptor (TCR) specificities are related to AD, in both the brain and blood of AD patients. It has also been shown that an increased response by antigen-experienced T-cells in the blood and cerebrospinal fluid of patients is affected by age-related neurodegeneration, noting an interesting TCR specificity for Epstein Barr virus antigens. Further, it was also noted that there is an association of anti-β-amyloid antibodies with cognitive decline. Interestingly, genetic variants are associated with altered regulation of macrophage specific genes, raising questions about roles for antigen presenting cells, as part of the role of the adaptive immune system in the development of AD.

Appreciating a potential role for the adaptive immune system in AD, it has been considered that modification of the immune response using strategies such as vaccination and antibody-therapy against known antigens, such as β-amyloid, has disease-modifying processes for AD. However, the previous widely assessed vaccination trial, where β-amyloid was the intended target, was terminated early as a result of the adaptive immune receptor (IR) response leading to meningoencephalitis in some patients. Other vaccination trials with both β-amyloid and Tau are underway but concerns over safety and effectiveness remain. For example, the FDA approved, amidst controversy, treatment with an antibody, aducanumab, targeting β-amyloid.

In sum, a further understanding of the role of the adaptive IRs in AD is needed. Thus, TCR recombination reads were characterized from blood- and brain-derived, AD patient exome (WXS) files, including an approach that employs a chemical complementarity assessment for CDR3s and candidate antigens designed for the big data setting. Results indicated, among other things, a correlation of higher AD+ patient Braak stages with increased chemical complementarity scores for both the blood- and brain-derived TRA CDR3s and Tau.

### Methods

Alzheimer's disease sequencing project (ADSP) WXS files; and biospecimen and survival data files. AD WXS files were downloaded from the ADSP Amazon Web Services (AWS) S3 bucket to this research group's AWS bucket in binary alignment map (BAM) format with authorization via database of genotypes and phenotypes (dbGaP) approved project 23565 (ADSP project ID: 10229). The download protocol is at dss.niagads.org/documentation/. To download only the WXS file slices needed for recovery of the IR recombination reads, an original script freely available at github.com/thudaUSF/ADSP-dl-process was used. This script generates a unique URL for each WXS file using boto3, which allows SAMtools, an S3-aware alignment reader, to download and process a section of the aligned data (e.g. the 930 kbp region of TRA, on chromosome 14), without having to download the full WXS file. The majority of samples were derived from blood or brain and from either Alzheimer's cases or controls. These samples were taken at multiple sites and sequencing centers, where steps were taken to reduce variance in data. Notably, each case was associated with only one blood or brain sample, i.e., there are no case-matched blood and brain samples in the dataset. This report utilizes only the blood or brain WXS samples for Alzheimer's cases (AD+) that have a Braak stage, representing a staging mechanism based on histological features, including the location of brain lesions and density of neurofibrillary tangles and neuropil threads. These stages progress from 0, transentorhinal (I-II), limbic (III-IV), and end with isocortical stages (V-VI). This staging process relies on a neuropathologist's opinion and above algorithm, blinded to age and other clinical data. Braak staging was chosen as the dependent variable, as it has been that Braak stages have correlated well with clinical symptoms of AD. Such clinical symptoms include cognitive measures, such as MMSE scores and cognitive measures, but also other behavioral symptoms beginning with symptoms such as agitation, anxiety, and depression in stages I-II and culminating in cognitive decline in stages V-VI. Thus, the dataset used includes 1113 blood cases and 1552 brain cases, for a total of 2665 cases with WXS files and an associated Braak stage. The file used for downloading all of the WXS files, termed the sample file, was created by combining information in the manifest and phenotype files obtained from the ADSP AWS bucket.

Obtaining the adaptive IR recombination reads. The IR recombination reads were retrieved from the WXS files for four T-cell receptor genes (TRA, TRB, TRD, TRG), and three immunoglobulin genes (IGH, IGK, IGL) for AD+ blood and brain WXS files. Only IR recombination reads with both a verifiable V- and J-gene segment ID on one read were used. This was done using a previously and extensively described algorithm and script, freely available at github.com/bchobrut-USF/blanck__group, with a container version at hub.docker.com/r/bchobrut/vdj. In both cases, a readme file is available at the web links. Briefly, three processes were applied to obtain and further evaluate all IR recombination reads: (i) a low-stringency search of the reads constituting the WXS files, using 10-mers that matched germline IR, V- and J-nucleotide sequences in multiple places in the V- and J-gene segments; (ii) a higher stringency search that required a 90% match of 19 nucleotides to a V- and J-gene segment on one read; and (iii) translation of the gene segment recombination junction, i.e., the CDR3 region, and retention of only those IR recombination reads that lacked a stop codon or an out-of-frame joining sequence. The following sections of the WXS files were targeted based on the hg38 version of the reference genome: TRA: chr14:21621904-22552132; TRB: chr7:142299011-142813287; TRG: chr7:38240024-38368055; TRD: chr14:22422546-22466577; IGH: chr14:105586437-106879844; IGK: chr2:88857361-90235368; IGL: chr22:22026076-22922913.

Identifying the single value physicochemical parameters of the TRA CDR3s associated with Braak stages. Utilizing TRA CDR3 amino acid (AA) sequence data from the above brain and blood samples, single value physicochemical parameters, for example, aromaticity, fraction-negative, and secondary-structure, were assessed using the algorithms and a script available at github.com/bchobrut-USF/blanck_group, package B. An Excel pivot table was used to obtain the average of each physicochemical parameter for the TRA CDR3 AA sequences representing each case. The analyses that linked the case IDs, based on physicochemical parameters, to the Braak stages were conducted using an original script available at github.com/thudaUSF/ADSP-dl-process. This script utilized both Python and rpy2 to first align cases with a Braak stage, then perform a Kruskal-Wallis (KW) test by ranks to determine whether the distribution of a physicochemical parameter is the same among the Braak stage groups. Samples with Braak stages III and IV were merged into one group for analyses due to sample size constraints. For all KW tests, the post-hoc Dunn test was performed with the Bonferroni correction.

Establishment of CDR3 homology groups. CDR3 homology groupings were based on calculations using the chemical complementarity scoring algorithms, which were designed for matching CDR3s with candidate antigens. By inverting the sign, positive or negative, for an electrostatic complementarity score, the complementarity scoring process becomes effectively a homology grouping process, referred to as an Electrostatic group. For this example, this process also included a separate homology grouping process based on Uversky hydropathy values, to obtain homology groups based on hydrophobicity (termed, Hydro group). The process also creates homology groups based on a combination of electrostatic and hydrophobic CDR3 AAs (termed, Combo group). Then, a distance matrix of homology grouping scores for all distinct TRA CDR3 domains in the dataset was established using the DistanceMatrix function of the skbio python package(scikit-bio.org/docs/0.5.2/generated/generated/skbio.stats.distance.DistanceMatrix.html). The phylogenetic grouping of CDR3s was performed using the distance matrix input and the neighbor joining algorithm of the skbio.tree python package (scikit-bio.org/docs/0.5.2/generated/skbio.tree.nj.html) followed by the Tree algorithm of the ete3 python package (etetoolkit.org). Duplicate CDR3s were removed prior to input, to minimize inclusion of any public CDR3s in the analyses, leaving 515 unique blood-derived cases and 89 unique brain-derived cases. The chemical homology groups for AD+ blood and brain TRA CDR3s were associated with the case ID's Braak stage, and the Braak stage for the case IDs in the chemical homology group were compared to the Braak stage for the case IDs not in the homology group, referred to as the “out-group”, using the Mann-Whitney U (MW) test (Results). The homology grouping was performed using an original web tool, adaptivematch.com, which is publicly accessible and has instructions for preparing input files.

Chemical complementarity scoring. The chemical complementarity scoring involved a sliding window approach to assess multiple alignments of a CDR3 and a candidate antigen with regard to chemical features. The mp4 file that provides a user-friendly video of the alignment and chemical complementarity scoring process is included. For any given CDR3-candidate antigen pair, the highest calculated, chemical complementarity score was used in subsequent analyses. This process was facilitated by the use of the web tool, adaptivematch.com. For this example, Tau was first matched, using the adaptivematch.com web tool, as a whole protein sequence, with blood and brain TRA CDR3s, for the chemical complementarity scoring. Then, Tau was split into approximately seven equal segments, and the chemical complementarity of the CDR3s with each segment of Tau was determined using the same electrostatic, hydrophobic, or combo calculations referenced above.

### Results

Single value physicochemical assessments for the brain, AD+ TRA CDR3s. Adaptive IR reads were mined (Methods) from the WXS files representing 1113 blood cases and 1552 brain cases, and the resulting IR recombination read counts are tallied in Table 1. Three thousand, nine hundred seventy-seven TRA CDR3s from blood were recovered, with at least one CDR3 from 46.36% of the 1113 blood cases. One hundred sixty-three TRA CDR3s from brain were recovered, with at least one CDR3 from 6.06% of the 1552 brain cases. TRA recombination reads represented the greatest number of recoveries for both blood and brain datasets, compared to recoveries of recombination reads representing the other IRs (TRB, TRC, TRD, TRG, IGH, IGK, IGL). The Braak stage co-variates for the cases representing the TRA recombination read recoveries from brain and blood are in Tables 2 and 3, respectively. The subsequent analyses largely focused on the TRA CDR3 reads.

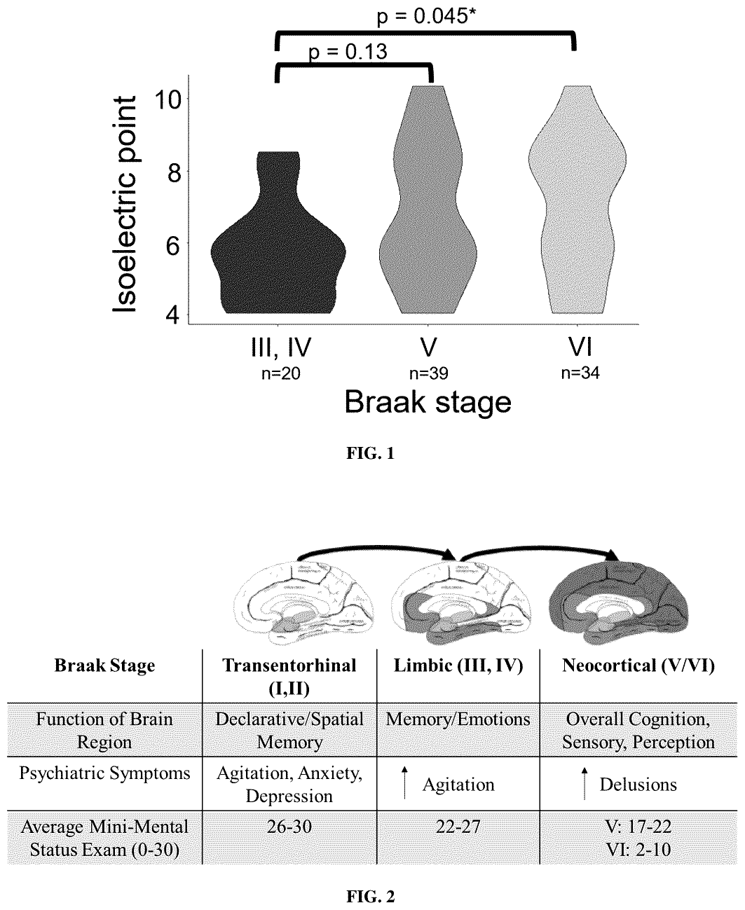

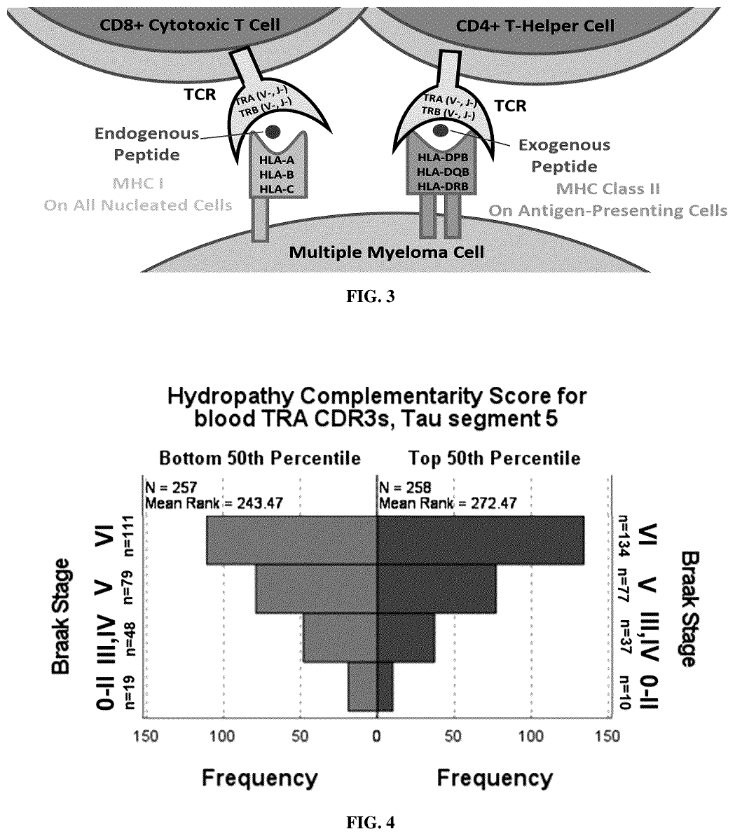

The single value physicochemical parameters for the TRA CDR3s were tested and shown to be associated with the histological Braak stages, which positively correlate with the severity of the disease. Results indicated that higher isoelectric values, for the TRA CDR3 AA sequences recovered from the brain WXS files, were associated with the higher Braak stages (Table 4, FIG. 1). Additional single value physicochemical parameters were similarly assessed, and notably, physicochemical parameters for the brain TRA CDR3 AAs related to isoelectric point were consistent with higher isoelectric point values being associated with higher Braak stages.

TRA CDR3 AA sequence homology grouping. Given the above conclusion of TRA CDR3 AA sequences having similar single value physicochemical features associated with higher Braak stages, a more extensive AA sequence was employed, and chemical homology grouping process for the TRA CDR3 AA sequences was recovered from the AD+ WXS files (Methods; adaptivematch.com). This approach led to the identification of groups of TRA CDR3 AA sequences that had a specific level of homology based on electrostatic charge, based on hydrophobicity, or based on the integration of both electrostatic charge and hydrophobicity in forming the homology group, i.e., in this latter case, there was an algorithmic incorporation of both chemical features in establishing the homology group (Methods). Results indicated that multiple homology groups representing both blood- and brain-derived TRA CDR3 AA sequences were associated with the distinct Braak stages (Table 5). These homology groups ranged, in the percentage of cases contributing to the homology group, from 1-26% of the collection of cases representing the TRA CDR3 AA sequence recoveries. While some TRA CDR3 AA sequence homology groups were associated with a higher Braak stage, and therefore a worse clinical condition, such as the Hydro homology group 547 (Table 5), other homology groups were associated with a lower Braak stage. The TRA CDR3 AA sequences for two examples of the homology groups, associated with lower Braak stages and representing blood- and brain-derived WXS files, are listed in Table 6.

Chemical complementarity scores for TRA CDR3s with Tau, as a whole protein, and for seven segments of the Tau protein. A CDR3-candidate antigen chemical complementarity scoring algorithm was applied using the blood- and brain-derived TRA CDR3s and the entire Tau protein sequence. A correlation between the Combo complementarity score and the higher Braak stages was identified using brain derived TRA CDR3s (Methods) (Table 7). Next, the Tau protein sequence was divided into seven segments to assess potentially more specific TRA CDR3-Tau chemical complementarity. Results indicated that the upper 50^{th} percentile for the Combo complementarity scores for brain, TRA CDR3s and Tau protein segment 7 was associated with the higher Braak stages, while the lower 50^{th} percentile, Combo complementarity scores for Segment 7 was associated with the lower Braak stages (p=0.044) (Table 8). For the blood-derived, TRA CDR3s, the Hydro complementarity score calculation (Methods) indicated an association of the TRA CDR3-Tau segment, chemical complementarity with the Braak stage. Thus, for Tau Segments 2 through 6, there was an association of a higher Hydro complementarity score with the higher Braak stages (Table 9).

Analysis of single value physicochemical parameters for the blood, AD+ TRG CDR3s. As previously mentioned, it has been noted that a role of γδ T cells in neuroinflammation exists and also noted that a particular TRG clonotype repertoire in AD patients exists. To further characterize AD+ TRG CDR3 AAs , the single value physicochemical parameters of the TRG CDR3 AA sequences from both blood and brain samples were analyzed. Results indicated that the higher isoelectric point values for the TRG CDR3 AA sequences recovered from the blood WXS files were associated with the higher Braak stages (Table 10). Moreover, a lower molecular weight for bloodborne IGH CDR3s was associated with the higher Braak stages (Table 10).

### Discussion

This is the first report of IR recombination reads obtained via mining AD+ blood or brain genomics files. Taken together, the above results indicate an opportunity to establish immunogenomics parameters for risk stratification in Alzheimer's disease. Specifically, the most important part of TRA for antigen binding, the CDR3, varies with regard to the chemical features of its AAs, and subcategories of this variation can be linked to different Braak stage categories. Furthermore, an established CDR3-candidate antigen chemical complementarity scoring algorithm, that has been used to identify differences in immune marker gene expression and survival probabilities in the cancer setting and is specifically applicable in a big data setting has been indicated by TRA CDR3-Tau chemical complementarity as associated with a higher Braak stage and thereby a worse clinical condition. No such chemical complementarity associations were detected for the β-amyloid peptide.

The recovery of adaptive IR reads from the brain was quantitatively modest, in comparison to certain cancer samples, such as melanoma. However, more robust IR recoveries for blood samples do indicate the application of immunogenomics parameters using blood derived IR reads, e.g., the association of high TRA CDR3-Tau chemical complementarity and higher Braak stages, when using TRA recombination reads obtained from blood (Table 9). Again, this indicates blood-based immune parameters associated with Alzheimer's features or occurrence. And importantly, the linkage of blood-based immune parameters with cognitive decline is consistent with immunological dynamics representing distant organs and systemic features, e.g., microbiome immunology features, being associated with cognitive decline.

The data reported here indicate an opportunity for biomarker development regarding disease severity, using a precise, quantitative representation of chemical interaction. A biomarker such as the TRA CDR3-Tau chemical complementarity score based on blood TRA recombination is particularly useful, given the noninvasive access to a blood sample. Furthermore, with such a noninvasive measurement, biomarkers can be identified in a relatively large cohort, i.e., a cohort large enough to assess persons at risk for Alzheimer's disease. These biomarkers additionally determine whether a high TRA CDR3-Tau complementarity score can indicate a transition to Alzheimer's disease, as opposed to establishing disease severity as the above-described associations of the blood-based, high TRA CDR3-Tau complementarity scores demonstrate (Table 9).

Furthermore, while this example has identified characteristics regarding the TCR repertoire for neuroinflammation in Alzheimer's disease, other neurodegenerative diseases including Parkinson's disease are investigated. In Parkinson's disease, it has been shown that γδ T-cells are increased and have demonstrated the presence of α-synuclein-specific T cells. In many cases where T-cell responses are at issue, this has indeed been the case, including infectious diseases, autoimmunity and cancer.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

TABLE 1
Numbers of adaptive immune receptor (IR) recombination reads,
representing productive recombinations, recovered from the
WXS files of the Alzheimer's disease (AD) dataset

| | | Unique | | Unique |
|---|---|---|---|---|
| | Blood | cases out of | Brain | cases out of |
| Immune | recovery | 1113 total | recovery | 1552 total |
| receptor | count | blood cases | count | brain cases |
| TRA | 3977 | 46.36% | 163 | 6.06% |
| TRB | 1447 | 31.72% | 51 | 1.74% |
| TRD | 63 | 2.88% | 3 | 0.13% |
| TRG | 2240 | 38.36% | 88 | 2.96% |
| IGH | 716 | 25.97% | 6 | 0.26% |
| IGK | 633 | 28.30% | 3 | 0.13% |
| IGL | 679 | 18.87% | 11 | 0.19% |

TABLE 2
Characteristics of AD+ cases with brain-sample WXS
samples with a TRA recovery, classified by Braak stage.

| | | | | | Apo E4 |
|---|---|---|---|---|---|
| Braak | | Age at Onset | | Sex | carrier |
| stage | N | Mean (SD) | | % Female | % |
| III-IV | 20 | 75.32 | (7.72) | 45 | 35 |
| V | 39 | 70.47 | (6.06) | 46.1 | 69.2 |
| VI | 34 | 69.35 | (5.66) | 47.1 | 79.4 |
| Total | 93 | 72.13 | (6.56) | 46.2 | 65.6 |
| p-value | | 0.01 | (KW) | 0.885 (MW) | 0.002 (MW) |

TABLE 3
Characteristics of AD+ cases with blood sample
WXS files with a TRA recovery, classified by Braak stage.

| | | | | | Apo E4 |
|---|---|---|---|---|---|
| Braak | | Age at Onset | | Sex | carrier |
| stage | N | Mean (SD) | | % Female | % |
| 0-II | 29 | 75.32 | (7.72) | 41.4 | 10.3 |
| III-IV | 85 | 75.7 | (7.66) | 47.1 | 31.8 |
| V | 157 | 73.7 | (7.60) | 39.5 | 49.7 |
| VI | 245 | 70.63 | (7.31) | 51.8 | 55.1 |
| Total | 516 | 72.61 | (7.72) | 46.7 | 47.1 |
| p-value | | <0.001 | (KW) | 0.078 (MW) | <0.001 (MW) |

TABLE 4
Isoelectric point (physiochemical) values for TRA CDR3s from brain-
derived AD+ samples correlated with Braak stage distinctions.

| | | | | | | | Braak | |
|---|---|---|---|---|---|---|---|---|
| | | | | | | | groups | |
| | | | | | | Associated | compared | |
| Alzheimer's | n | | | | | with a | in a Dunn | |
| brain or | (number | n | | | | higher | post-hoc | |
| blood as | of cases) | Braak | n | n | | (worse) | test | Post- |
| TRA | Braak | group | Braak | Braak | KW | Braak | (comparison | hoc |
| CDR3 | group | III, | group | group | p- | stage | across the | p- |
| source | 0-II | IV | V | VI | value | group | hyphen) | value |
| Brain tissue | 1 | 20 | 39 | 34 | 0.044 * | Increased | III, IV-V | 0.13 |
| | (dropped) | | | | | isoelectric | III, IV-VI | 0.045 * |
| | | | | | | point | V-VI | 1 |
| Blood | 29 | 85 | 157 | 245 | 0.53 | — | — | — |
| samples | | | | | | | | |

TABLE 5
Chemical homology groups representing the AA sequences of the blood and
brain-derived TRA CDR3s were associated with distinct Braak stages.

| | | Designation | | | |
|---|---|---|---|---|---|
| | | of outside- | | | |
| | | homology-group | | | |
| | | or in-homology- | Number of | | |
| Homology | | group for the | cases either | | Associated |
| grouping | | parameter | out or in | | with the |
| parameter | | indicated two | the chemical | Mann- | higher (worse) |
| and TRA | Homology | columns to the | homology | Whitney | Braak stage |
| CDR3 source | group | left | group | p-value | group |
| Hydro, blood | Group 547 | All remaining | 491 | 0.040 | In-group |
| | | TRA CDR3s | | | |
| | | Homologous | 24 | | |
| | | TRA CDR3s | | | |
| Hydro, brain | Group 39 | All remaining | 84 | 0.021 | Out-group |
| | | TRA CDR3s | | | |
| | | Homologous | 5 | | |
| | | TRA CDR3s | | | |
| Electrostatic, | Group 416 | All remaining | 379 | 0.025 | Out-group |
| blood | | TRA CDR3s | | | |
| | | Homologous | 136 | | |
| | | TRA CDR3s | | | |
| Electrostatic, | Group 43 | All remaining | 81 | 0.035 | Out-group |
| brain | | TRA CDR3s | | | |
| | | Homologous | 8 | | |
| | | TRA CDR3s | | | |
| Combo, blood | Group 61 | All remaining | 474 | 0.011 | Out-group |
| | | TRA CDR3s | | | |
| | | Homologous | 41 | | |
| | | TRA CDR3s | | | |
| Combo, brain | Group 33 | All remaining | 83 | 0.024 | Out-group |
| | | TRA CDR3s | | | |
| | | Homologous | 6 | | |
| | | TRA CDR3s | | | |

TABLE 6
TRA CDR3s in the indicated
homology in-groups from Table 5.

| Blood Group 61 CDR3s, | Brain Group 33 CDR3s, |
|---|---|
| representing the | representing the |
| combo grouping | combo grouping |
| parameter | parameter |
| CAETTLDENKFYF | CAFMTQWGGSQGNLIF |
| (SEQ ID NO: 1) | (SEQ ID NO: 42) |
| CALSEETGANSKLTF | CCGGGGGGSQGNLIF |
| (SEQ ID NO: 2) | (SEQ ID NO: 43) |
| CSEWWAAGNKLTF | CAGLNYGGSQGNLIF |
| (SEQ ID NO: 3) | (SEQ ID NO: 44) |
| CALSDIGGATNKLIF | CAASAFSSGSARQLTF |
| (SEQ ID NO: 4) | (SEQ ID NO: 45) |
| CAVSDGGGSNYKLTF | CAVNPHGSAGNMLTF |
| (SEQ ID NO: 5) | (SEQ ID NO: 46) |
| CATDASAAGNKLTF | CAVGNYGGSQGNLIF |
| (SEQ ID NO: 6) | (SEQ ID NO: 47) |
| CALSEAGGATNKLIF | |
| (SEQ ID NO: 9) | |
| CAEIPINDYKLSF | |
| (SEQ ID NO: 7) | |
| CATDATGGGNKLTF | |
| (SEQ ID NO: 10) | |
| CAVSEGSGGYQKVTF | |
| (SEQ ID NO: 11) | |
| CGADGTSGSARQLTF | |
| (SEQ ID NO: 8) | |
| CLVGESSSGTYKYIF | |
| (SEQ ID NO: 12) | |
| CGTEGITSGSRLTF | |
| (SEQ ID NO: 14) | |
| CAEAGGGYQKVTF | |
| (SEQ ID NO: 15) | |
| CAVEGTGGYQKVTF | |
| (SEQ ID NO: 16) | |
| CAVSDHSSGSARQLTF | |
| (SEQ ID NO: 17) | |
| CGEQGTGGFKTIF | |
| (SEQ ID NO: 18) | |
| CALGGENAGNNRKLIW | |
| (SEQ ID NO: 19) | |
| CAVSEIYNQGGKLIF | |
| (SEQ ID NO: 20) | |
| CAFADSTSGTYKYIF | |
| (SEQ ID NO: 21) | |
| CGEPLSGYNKLIF | |
| (SEQ ID NO: 13) | |
| CAYEAGGANSKLTF | |
| (SEQ ID NO: 22) | |
| CATDPSNQGGKLIF | |
| (SEQ ID NO: 23) | |
| CAVSENSGGYQKVTF | |
| (SEQ ID NO: 24) | |
| CALIEFSGGYNKLIF | |
| (SEQ ID NO: 25) | |
| CLVGDLNTGFQKLVF | |
| (SEQ ID NO: 26) | |
| CATDLIQGAQKLVF | |
| (SEQ ID NO: 27) | |
| CAMSEYGGATNKLIF | |
| (SEQ ID NO: 28) | |
| CAESHSGSARQLTF | |
| (SEQ ID NO: 29) | |
| CGTELTSGTYKYIF | |
| (SEQ ID NO: 30) | |
| CLVGDGSGNTGKLIF | |
| (SEQ ID NO: 31) | |
| CATDYGGATNKLIF | |
| (SEQ ID NO: 32) | |
| CVVSDGSGGYNKLIF | |
| (SEQ ID NO: 33) | |
| CAELLSGTYKYIF | |
| (SEQ ID NO: 34) | |
| CAGQDSGGSNYKLTF | |
| (SEQ ID NO: 35) | |
| CAVSDVTGGGNKLTF | |
| (SEQ ID NO: 36) | |
| CAVEGGTSYGKLTF | |
| (SEQ ID NO: 37) | |
| CSDALQGAQKLVF | |
| (SEQ ID NO: 38) | |
| CAEGSSNTGKLIF | |
| (SEQ ID NO: 39) | |
| CCAPDGSSNTGKLIF | |
| (SEQ ID NO: 40) | |
| CALSDGTGGGNKLTF | |
| (SEQ ID NO: 41) | |

TABLE 7
Higher Combo complementarity scores (CSs) for
AD+ brain TRA CDR3s and the Tau protein
(MAPT) were associated with higher Braak stages.

| | | | Associated |
|---|---|---|---|
| | Number | | with the |
| | of cases | | higher (worse) |
| Alzheimer's brain Combo | used for | MW | Braak stage |
| CS 50th percentile groups | correlation | p-value | group |
| Lower 50^{th} percentile, brain | 44 | 0.037 | Increased |
| TRA CDR3-full length Tau CSs | | | CS |
| Higher 50^{th} percentile, brain | 45 | | |
| TRA CDR3-full length Tau CSs | | | |

TABLE 8
Higher Combo complementarity scores (CSs) for AD+
brain TRA CDR3s and the Tau protein (MAPT) Segment
7 were associated with higher Braak stages.

| | | | Associated |
|---|---|---|---|
| | Number | | with the |
| | of cases | | higher (worse) |
| Alzheimer's brain Combo | used for | MW | Braak stage |
| CS 50th percentile groups | correlation | p-value | group |
| Lower 50^{th} percentile, brain TRA | 44 | 0.044 | Increased |
| CDR3-Tau Segment 7 Combo CSs | | | CS |
| Higher 50^{th} percentile, brain TRA | 45 | | |
| CDR3-Tau Segment 7 Combo CSs | | | |

TABLE 9
Higher Hydro complementarity scores (CS) for AD+
blood TRA CDR3s and the Tau Segments 2 through
6 were associated with higher Braak stages.

| | | | | Associated |
|---|---|---|---|---|
| | | | | with the |
| Tau | | | | higher (worse) |
| protein | Alzheimer's blood Hydro | Number | MW | Braak stage |
| segment | CS 50th percentile groups | of cases | p-value | group |
| 2 | Lower 50^{th} percentile blood | 257 | 0.026 | Increased |
| | TRA CDR3-Segment 2 | | | CS |
| | Upper 50^{th} percentile blood | 258 | | |
| | TRA CDR3-Segment 2 | | | |
| 3 | Lower 50^{th} percentile blood | 257 | 0.028 | Increased |
| | TRA CDR3-Segment 3 | | | CS |
| | Upper 50^{th} percentile blood | 258 | | |
| | TRA CDR3-Segment 3 | | | |
| 4 | Lower 50^{th} percentile blood | 257 | 0.032 | Increased |
| | TRA CDR3-Segment 4 | | | CS |
| | Upper 50^{th} percentile blood | 258 | | |
| | TRA CDR3-Segment 4 | | | |
| 5 | Lower 50^{th} percentile blood | 257 | 0.017 | Increased |
| | TRA CDR3-Segment 5 | | | CS |
| | Upper 50^{th} percentile blood | 258 | | |
| | TRA CDR3-Segment 5 | | | |
| 6 | Lower 50^{th} percentile blood | 257 | 0.020 | Increased |
| | TRA CDR3-Segment 6 | | | CS |
| | Upper 50^{th} percentile blood | 258 | | |
| | TRA CDR3-Segment 6 | | | |

TABLE 10
Physicochemical parameters of AD+ bloodborne TRG
and IGH CDR3s were associated with Braak stage distinctions.

| | | | | | | Associated | | |
|---|---|---|---|---|---|---|---|---|
| | n | | | | | with the | Braak | |
| | (Number | n | | | | higher | groups | |
| | of cases) | Braak | n | n | | (worse) | compared | Post- |
| Receptor and | Braak | group | Braak | Braak | KW | Braak | in Dunn | hoc |
| physicochemical | group | III, | group | group | p- | stage | Post-hoc | p- |
| parameter used | 0-II | IV | V | VI | value | group | test | value |
| Cases with TRG | 18 | 68 | 132 | 209 | 0.021* | Increased | 0-II-III, IV | 1 |
| recovery | | | | | | property | 0-II-V | 0.11 |
| Isoelectric | | | | | | | 0-II-VI | 0.10 |
| point | | | | | | | III, IV-V | 0.27 |
| | | | | | | | III, IV-VI | 0.35 |
| | | | | | | | V-VI | 1 |
| Cases with IGH | 61 | 64 | 88 | 74 | 0.018* | Decreased | 0-II-III, IV | 1 |
| recovery | | | | | | property | 0-II-V | 1 |
| Molecular | | | | | | | 0-II-VI | 1 |
| weight | | | | | | | III, IV-V | 0.47 |
| | | | | | | | III, IV-VI | 0.034* |
| | | | | | | | V-VI | 0.037* |

TABLE 11
Tau Protein Fragment Sequences

| Tau Antigen | Antigen Sequence |
|---|---|
| 1 | MAEPRQEFEVMEDHAGTYGLGD |
| | RKDQGGYTMHQDQEGDTDAGL |
| | KESPLQTPTEDGSEEPG |
| | (SEQ ID NO: 48) |
| 2 | SETSDAKSTPTAEAEEAGIGD |
| | TPSLEDEAAGHVTQARMVSKS |
| | KDGTGSDDKKAKGADGKT |
| | (SEQ ID NO: 49) |
| 3 | KIATPRGAAPPGQKGQANATR |
| | IPAKTPPAPKTPPSSGEPPKS |
| | GDRSGYSSPGSPGTPGSR |
| | (SEQ ID NO: 50) |
| 4 | SRTPSLPTPPTREPKKVAVVR |
| | TPPKSPSSAKSRLQTAPVPMP |
| | DLKNVKSKIGSTENLKHQ |
| | (SEQ ID NO: 51) |
| 5 | PGGGKVQIINKKLDLSNVQSK |
| | CGSKDNIKHVPGGGSVQIVYK |
| | PVDLSKVTSKCGSLGNIH |
| | (SEQ ID NO: 52) |
| 6 | HKPGGGQVEVKSEKLDFKDRV |
| | QSKIGSLDNITHVPGGGNKKI |
| | ETHKLTFRENAKAKTDHG |
| | (SEQ ID NO: 53) |
| 7 | AEIVYKSPVVSGDTSPRHLSN |
| | VSSTGSIDMVDSPQLATLADE |
| | VSASLAKQGL |
| | (SEQ ID NO: 54) |

What is claimed is:

1. A method of detecting a progression of Alzheimer's disease in a subject, comprising:
obtaining and isolating a nucleic acid sample from a biological sample derived from the subject;
sequencing a first nucleic acid sequence of a complementary determining region (CDR) of an antigen-binding protein and sequencing a second nucleic acid sequence of a misfolded or aggregated protein, or a fragment thereof, associated with Alzheimer's disease;
translating the first nucleic acid sequence into an amino acid sequence of the complementary determining region (CDR) of the antigen-binding protein and translating the second nucleic acid sequence into an amino acid sequence of the misfolded or aggregated protein, or a fragment thereof, associated with Alzheimer's disease;
determining a chemical complementarity score between the amino acid sequence from the CDR and the amino acid sequence of the protein associated with Alzheimer's disease; and
detecting the progression of Alzheimer's disease in the subject, wherein a first chemical complementarity score is higher in a first biological sample derived from the subject compared to a chemical complementarity score from a reference control, and wherein a second chemical complementarity score is higher in a second biological sample derived from the subject compared the first chemical complementarity score and the chemical complementarity score from the reference control.

2. The method of claim 1, wherein the chemical complementarity score represents an interaction between one or more amino acids of the CDR of the antigen-binding protein and one or more amino acids of the protein associated with Alzheimer's disease.

3. The method of claim 1, wherein a low chemical complementarity score indicates a low-level (transentorhinal) Braak stage of Alzheimer's disease.

4. The method of claim 1, wherein a high chemical complementarity score indicates a mid-level (limbic stage) or an advanced level (neocortical) Braak stage of Alzheimer's disease.

5. The method of claim 3, wherein the low-level Braak stage comprises Braak stage I or Braak stage II.

6. The method of claim 4, wherein the mid-level Braak stage comprises Braak stage III or Braak stage IV, and wherein the advanced-level Braak stage comprises Braak stage V or Braak stage VI.

7. The method of claim 1, wherein the antigen-binding protein comprises a T cell receptor alpha (TRA) chain or a T cell receptor beta (TRB) chain.

8. The method of claim 1, wherein the misfolded or aggregated protein associated with Alzheimer's disease comprises a tau protein, a fragment thereof, or a related protein.

9. The method of claim 1, comprising administering a therapeutic composition after detecting the progression of Alzheimer's disease in the subject, wherein the therapeutic composition comprises donepezil, galantamine, rivastigmine, memantine, lecanemab, aducanumab, or a related composition.

10. The method of claim 1, wherein the biological sample comprises a blood sample.

11. The method of claim 1, wherein the subject is a human.

12. A method of treating or preventing a progression of Alzheimer's disease in a subject in need thereof, the method comprising:
obtaining and isolating a nucleic acid sample from a biological sample derived from the subject;
sequencing a first nucleic acid sequence of a complementary determining region (CDR) of an antigen-binding protein and sequencing a second nucleic acid sequence of a misfolded or aggregated protein, or a fragment thereof, associated with Alzheimer's disease;
translating the first nucleic acid sequence into an amino acid sequence of the complementary determining region (CDR) of the antigen-binding protein and translating the second nucleic acid sequence into an amino acid sequence of the misfolded or aggregated protein, or a fragment thereof, associated with Alzheimer's disease;
determining a chemical complementarity score between the amino acid sequence from the CDR and the amino acid sequence of the protein associated with Alzheimer's disease;
detecting the progression of Alzheimer's disease in the subject, wherein a first chemical complementarity score is higher in a first biological sample derived from the subject compared to a chemical complementarity score from a reference control, and wherein a second chemical complementarity score is higher in a second biological sample derived from the subject compared the first chemical complementarity score and the chemical complementarity score from the reference control; and
administering a therapeutic composition to slow the progression of Alzheimer's disease in the subject.

13. The method of claim 12, wherein the chemical complementarity score represents an interaction between one or more amino acids of the CDR of the antigen-binding protein and one or more amino acids of the protein associated with Alzheimer's disease.

14. The method of claim 12, wherein a low chemical complementarity score indicates a low-level (transentorhinal) Braak stage of Alzheimer's disease.

15. The method of claim 12, wherein a high chemical complementarity score indicates a mid-level (limbic stage) or an advanced level (neocortical) Braak stage of Alzheimer's disease.

16. The method of claim 14, wherein the low-level Braak stage comprises Braak stage I or Braak stage II.

17. The method of claim 15, wherein the mid-level Braak stage comprises Braak stage III or Braak stage IV, and wherein the advanced-level Braak stage comprises Braak stage V or Braak stage VI.

18. The method of claim 12, wherein the antigen-binding protein comprises a T cell receptor (TCR) alpha (TRA) chain or a TCR beta (TRB) chain.

19. The method of claim 12, wherein the misfolded or aggregated protein associated with Alzheimer's disease comprises a tau protein, a fragment thereof, or a related protein.

20. The method of claim 12, wherein the therapeutic composition comprises donepezil, galantamine, rivastigmine, memantine, lecanemab, aducanumab, or a related composition.
